(19) United States
(12) Patent Application Publication
(10) Pub. No.: US 2009/0165795 A1
(43) Pub. Date: Jul. 2, 2009

(54) METHOD AND APPARATUS FOR RESPIRATORY THERAPY

(75) Inventors: Hossein Nadjafizadeh, Villers-Les-Nancy (FR); Pascal Nicolazzi, Gondreville (FR); Veronique Grillier-Lanoir, Besancon (FR); Bruno Flemmich, Jezainville (FR); Julien Gentner, Chaligny (FR)

Correspondence Address: NELLCOR PURITAN BENNETT LLC;ATTN: IP LEGAL, 60 Middleton Avenue, North Haven, CT, 06473, US

(73) Assignee: Nellcor Puritan Bennett LLC, Boulder, CO (US)

(21) Appl. No.: 12/337,514

(22) Filed: Dec. 17, 2008

Related U.S. Application Data
(60) Provisional application No. 61/018,126, filed on Dec. 31, 2007.

(51) Int. Cl. A61M 16/00

(52) U.S. Cl. 128/204.18; 128/204.26; 128/204.23

(57) ABSTRACT

The disclosure provides a method for delivering pressurized gas to an airway of a subject. The disclosure also provides a system for delivering pressurized gas to an airway of a subject. The disclosure further provides a computer-readable storage medium containing a set of instructions executable on a processor that include routines to monitor a respiratory flow rate from a subject, generate a filtered flow and a dynamic flow from the respiratory flow rate, and control a gas generator connected to a breathing device.

### RELATED APPLICATION

This application claims priority from U.S. Provisional Application No. 61/018,126, filed, Dec. 31, 2007, which is hereby incorporated by reference herein in its entirety.

### TECHNICAL FIELD

The present disclosure relates generally to the field of respiratory therapy and more particularly to the application of respiratory therapies.

### BACKGROUND

Sleep apnea occurs when a person stops breathing during sleep. An apnea may generally be defined as the cessation of airflow for a period of time, e.g., more than 10 seconds. Apneas may lead to decreased blood oxygenation and thus, to the disruption of sleep. With some apneas, e.g., central apnea, the subject's airway is open however, the subject is not attempting to breathe. Conversely, with other apneas, the airway is closed, The airway may also be partially obstructed (i.e., narrowed). This also leads to decreased ventilation, decreased blood oxygenation, and/or disturbed sleep.

A common form of treatment for apnea is the administration of Continuous Positive Airway Pressure (hereinafter “CPAP”). Effective CPAP treatment may act as a pneumatic splint of the airway by the provision of a constant positive pressure usually in the pressure range of about 4 to about 20 cmH2O. Another form of treatment for apnea, is the administration of bi-level treatment. With bi-level treatment, one constant pressure level is provided during inhalation and a second constant pressure level, generally a lower pressure level, is provided during exhalation. With both treatments, increased pressure is supplied to the airway of the subject by a motor driven blower whose outlet supplies air via a delivery hose and mask to the subject's airway, e.g., via the subject's nose, mouth, or both (nose and mouth). An exhaust port may be provided in the mask and/or the delivery tube proximate the mask. The mask may take the form of a nose, mouth and/or face mask or nasal prongs, pillows or cannulae.

In many cases, subjects who experience sleep apnea also experience a significant narrowing of the upper airways during the latter part or period of exhalation and in the upper airways at the end of exhalation. In addition, airway occlusion or narrowing at the end of exhalation often precedes an apneic event and airway resistance during exhalation also increases prior to apneic events. As a result, ordinary CPAP therapy may make it difficult for a subject to exhale because the exhalation is resisted by a continuous positive pressure of air. Current treatments for this problem include monitoring a subject's airflow and adjusting the applied pressure breath-by-breath so that a variable pressure is applied to a subject during exhalation. The pressure that is applied during exhalation is lower than the CPAP pressure and varies on a breath-by-breath basis depending on the subject's airflow. Under current treatments, the pressure forms a reverse bell curve, where the applied pressure is gradually lowered when exhalation begins, reaches a minimum point at the middle of exhalation, and then is raised gradually so that the CPAP level is reached when inhalation begins. Although this treatment lowers the applied pressure resisting a subject's exhalation, exhalation by a subject during the first part of exhalation may still be more difficult than necessary. This is because the decrease in the applied pressure during exhalation under current treatments is gradual and the subject must exhale against a pressure that is higher that the minimum pressure point that will be applied during all but an instantaneous moment of exhalation.

### SUMMARY

In accordance with the present disclosure, systems and methods for detecting respiratory events and applying improved respiratory therapies are provided. According to one embodiment, a method for delivering pressurized gas to an airway of a subject is disclosed. The method may include applying a constant pressure at a level, e.g., a predetermined level, less than an effective pressure, e.g., therapeutic pressure, at approximately the beginning of exhalation until approximately the nadir of exhalation, raising the applied pressure to an effective pressure at rate, e.g., a predetermined rate, beginning at or approximately at the nadir of exhalation until the beginning or until approximately the beginning of inhalation, and applying pressure at an effective pressure during inhalation.

According to another embodiment, a system for delivering pressurized gas to an airway of a subject is disclosed. The system may include a gas source, e.g. a blower, a flow sensor connected for monitoring, preferably continuously monitoring, e.g., via a pilot tube or via any other methodology, a respiratory flow rate from the subject, a low pass filter operable to generate at least one of and preferably both a filtered flow and a dynamic flow from the respiratory flow rate, a pressure controller connected to the gas source, e.g., a gas generator, operable to control pressure levels applied from the gas source, and an event detection device, e.g., an event detector, connected to the pressure controller wherein the event detection device is preferably operable to: detect the beginning of exhalation in the subject, provide a signal to the pressure controller to apply a constant pressure at a level, e.g., a predetermined level, less than an effective or therapeutic pressure, detect the nadir or approximately the nadir of exhalation in the subject; send a signal to the pressure controller to raise the applied pressure to an effective pressure at a rate, e.g., a predetermined rate; detect approximately the beginning or the beginning of inhalation; and send a signal to the pressure controller to apply pressure at an effective pressure.

According to another embodiment, a computer-readable storage medium containing a set of instructions executable on a processor is disclosed. The set of instructions may include a routine operable to monitor, e.g., continuously, a respiratory flow rate from a subject, and a routine operable to control a motor, blower, or pump connected to apply a constant pressure at a level, preferably a predetermined level less than an effective pressure at the beginning or approximately the beginning of exhalation until the nadir (or approximately the nadir) of exhalation; raise the applied pressure to an effective pressure, e.g., a therapeutic pressure, at a rate, preferably a predetermined rate beginning at or about the nadir of exhalation until at or about the beginning of inhalation; and apply pressure at an effective or therapeutic pressure during inhalation.

According to another embodiment, a system for delivering pressurized gas to an airway of a subject is disclosed. The system may include a gas source means operable to deliver pressurized gas to the subject, a flow sensing means operable to continuously monitor a respiratory flow rate from the subject, a low pass filter means operable to generate at least one of and preferably both of a filtered flow and a dynamic flow from the respiratory flow rate, a pressure controller means connected to the gas source operable to control pressure levels applied from the gas source, and an event detection means connected to the pressure controller means wherein the event detection means may be operable to: detect the beginning or the approximate beginning of exhalation in the subject, send a signal to the pressure controller means to apply a constant pressure at a level, e.g., a predetermined level, less than an effective pressure, detect the nadir (or an approximate nadir) of exhalation in the subject, send a signal to the pressure controller means to raise the applied pressure to an effective pressure, e.g., a therapeutic pressure at a rate, e.g., a predetermined rate, detect the beginning or approximate beginning of inhalation; and send a signal to the pressure controller means to supply an effective or therapeutic pressure.

### BRIEF DESCRIPTION OF THE DRAWINGS

Some embodiments of the disclosure may be understood by referring, in part, to the following description and the accompanying drawings, in which like reference numbers refer to the same or like parts, and wherein:

FIG. 1 is a diagram of a breathing apparatus according to an embodiment of the present disclosure;

FIG. 2 is a flow diagram according to an embodiment of the present disclosure; and

FIG. 3 is a graphical illustration according to an embodiment of the present disclosure.

### DETAILED DESCRIPTION

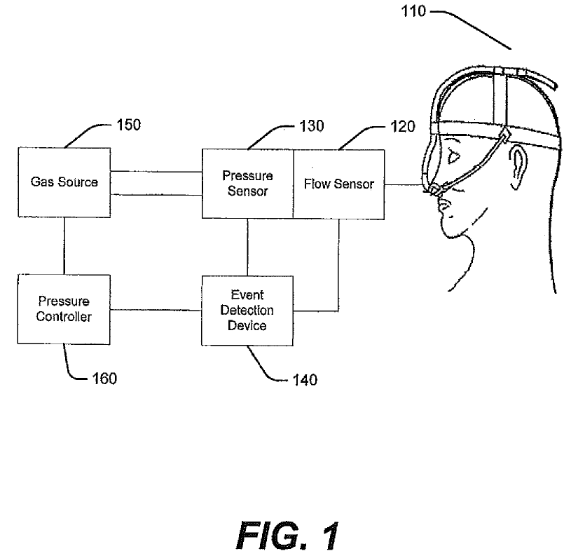

Selected embodiments of the disclosure may be understood by reference, in part, to FIGS. 1-3, wherein like numbers refer to same and like parts.

In general, the present disclosure describes methods and apparatuses for applying positive pressure respiratory therapy and for decreasing and maintaining a low and fixed applied pressure level during a first part or first period of exhalation. The near immediate application of a relief pressure, preferably a fixed relief pressure value at or during the beginning of exhalation, as opposed to the gradual application of a reduction in pressure, offers more immediate improvement to a subject's ability to exhale and reduces the likelihood of the occurrence of an apneic event. According to one example of the present disclosure, instead of applying respiratory therapy by gradually lowering and raising the pressure between exhalation and inhalation, the apparatus of FIG. 1 may apply positive pressure respiratory therapy by maintaining a low and preferably fixed applied pressure level during a first part or first period of exhalation, raising the applied pressure to an effective pressure at a rate, preferably a predetermined rate starting at or about at the peak of exhalation, and applying an effective pressure level during inhalation.

FIG. 1 illustrates an apparatus and system according to one embodiment of the present disclosure. In reference to FIG. 1, an apparatus of the present disclosure may include a mask 110, a flow sensor 120, a pressure sensor 130, an event detection device 140, a gas source 150, and a pressure controller 160. Note, the pressure controller of the present disclosure may control the gas source, e.g., a blower or blowers, one or more valves, or a combination of these. The apparatus of FIG. 1 may be used to both detect a subject's respiratory phases and to apply respiratory therapies based on the subject's respiratory phases.

The apparatus of FIG. 1 may be used as a respiratory apparatus in multiple modes, for example, two modes. One mode, called continuous positive air pressure mode (CPAP), provides a constant pressure level to a subject. This pressure level may be determined and set by a physician and is often used to provide respiratory therapy to a subject. In some embodiments, the device of FIG. 1 may operate in “bi-level” mode, which provides one constant pressure level during inhalation and provides a second constant generally lower pressure level during exhalation. The pressure may be provided to the subject via mask 110 and may be created by gas source 150. As noted, gas source 150 may be a blower or any air pump or other suitable device operable to provide gas. The pressure level may be controlled by pressure controller 160 connected to gas source 150. Pressure controller 160 may be any variety of analog or digital switches, actuators, valves, or control devices operable to achieve a desired pressure level from a gas source. Pressure controller 160 may be connected to an event detection device 140, discussed more fully below, and may be controlled based on the respiratory phases detected by event detection device 140. Event detection device 140 may be connected to pressure sensor 130 and/or to flow sensor 120. Pressure sensor 130 may detect a pressure within mask 110 and flow sensor 120 may detect the airflow from or to the mask 110. Pressure and/or flow information may be used by the event detection device 140 to, e.g., detect respiratory phases and control via pressure controller 160 gas source 150.

In the apparatus of FIG. 1, flow sensor 120 may, e.g., be a pilot tube and may monitor, e.g., continuously monitor, an instantaneous flow or flow rate signal. Flow sensor 120 may be located in conjunction with the gas source, in the delivery tube, and/or in the mask. The flow signal may, for example, be used to determine the different phases in a subject's breathing cycle. For instance, flow may be used to determine if the subject has begun inhalation, if the subject has reached the peak of inhalation, if the subject has begun exhalation, and/or if the subject has reached the peak of exhalation. The different phases in a subject's breathing cycle may be used as events or triggers to apply appropriate pressure levels to the subject according to some embodiments of the present disclosure. The flow and/or flow rate may be used to determine these phases in the following manner. The flow may include a subject's flow and may also include leak flow discharged through the mask 110 exhaust port with potential undesirable leakage from the subject's interface with the mask 110 or due to partial mouth breathing. The total flow signal may be processed by the event detection device 140 to generate several signals. First, a filtered flow signal may be generated from the instantaneous flow signal by the use of a filter, such as a low pass filter. The filtered flow signal may be used to represent the low frequency component of the total flow signal. The filtered flow signal may be used to detect the inhalation and exhalation phases in the subject's respiratory cycle based on systems and methods known in the art. Second, a dynamic flow signal may be determined by removing the filtered flow signal from the total flow signal. The dynamic flow signal may be used to represent the high frequency component of the total flow signal. The dynamic flow signal may be used to determine the exhalation phase nadir based on systems and methods known in the art.

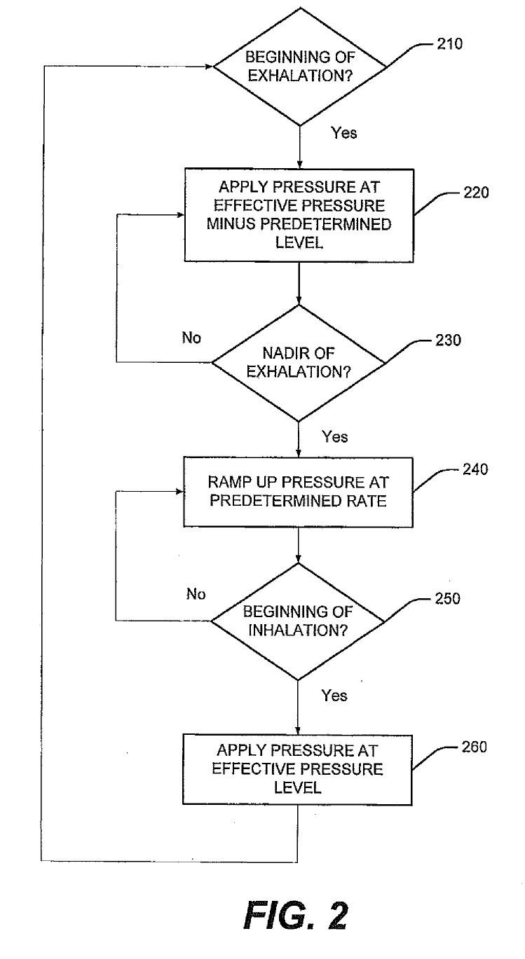

FIG. 2 illustrates the application of breathing therapy according to one embodiment of the present disclosure. According to FIG. 2, a constant positive effective pressure may be applied to a subject though mask 110 during inhalation, i.e., from the beginning or about the beginning of the subject's inhalation phase 250 until the beginning or about the beginning of the subject's exhalation phase 210. The phases may be determined by event detection device 140. This higher pressure applied during inhalation may improve the subject's ability to inhale and reduce the chance that an apneic event may occur. The effective pressure may be prescribed by a physician or other clinician, respiratory therapist, or the like and may represent the appropriate therapy for a subject to help alleviate sleeping abnormalities, For instance, the effective pressure level may be in the range of about eight to about twelve cmH2O. The effective or therapeutic pressure may be any pressure determined to be effective for treating a subject's breathing abnormalities during sleep. Once the beginning of the subject's exhalation phase is detected by event detection device 140 at point 210, a lower pressure, a relief pressure, or other pressure may be nearly instantaneously applied at step 220 to the subject via mask 110 at a value, e.g., a fixed value, from the beginning or approximately the beginning of exhalation 210 until approximately the nadir or at the nadir of exhalation 230. The near immediate application of a relief pressure at the start or beginning of exhalation, as opposed to the gradual reduction to a relief pressure, offers a more immediate improvement to a subject's ability to exhale. This improvement may also reduce the frequency and/or likelihood of an apneic event occurring in a subject. The pressure may come from gas source 150 and pressure levels may be controlled by pressure controller 160. The relief pressure may be the therapeutic pressure minus a predetermined pressure level value, for instance, minus three cmH20. The pressure decrease to the relief pressure, may occur in less than five-hundred milliseconds, or four-hundred milliseconds, and preferably occurs in less than three-hundred milliseconds when the therapeutic pressure is ten cmH2O and the predetermined exhalation comfort pressure level is three cmH2O. The time period required to arrive at the predetermined exhalation comfort pressure may vary depending upon the therapeutic pressure and the particular patient. The predetermined exhalation comfort pressure level may be, e.g., three cmH2O, two cmH2O, 1 cmH2O or less, or the therapeutic pressure minus any pressure level value determined to ease a subject's exhalation.

For example, in the event a subject's effective pressure is determined to be eight cmH2O and the predetermined pressure level is determined to be three cmH2O, a fixed relief pressure value of five cmH2O may be applied to the subject via mask 110 and gas source 150 from nearly the beginning of exhalation 210 until the nadir or approximately the nadir of exhalation 230, as determined by event detection device 140. Once a subject has reached the nadir or approximately the nadir of exhalation 230, the pressure applied to the subject may be raised at a rate, e.g., preferably a predetermined rate such that the effective pressure may be reached when the subject begins the inhalation phase of breathing at step 250. The predetermined rate may preferably be proportional to the subject's flow rate. The steps of FIG. 2 may apply equally to breathing therapies applied in CPAP or bi-level mode. In general, in bi-level mode, one constant pressure is applied during a subject's inhalation and a lower constant pressure is applied during a subject's exhalation, Under some embodiments of the present disclosure applying bi-level therapies, an overall lower fixed pressure may be applied during the subject's exhalation phase and the pressure applied during the subject's inhalation phase remains the same. According to one bi-level embodiment of the disclosure, a first inhalation pressure is applied during inhalation, a second exhalation end pressure is applied at the end of exhalation, and a third beginning of exhalation pressure is applied at the beginning of exhalation. The third beginning of exhalation pressure is lower than the first inhalation pressure and lower than the second end exhalation pressure.

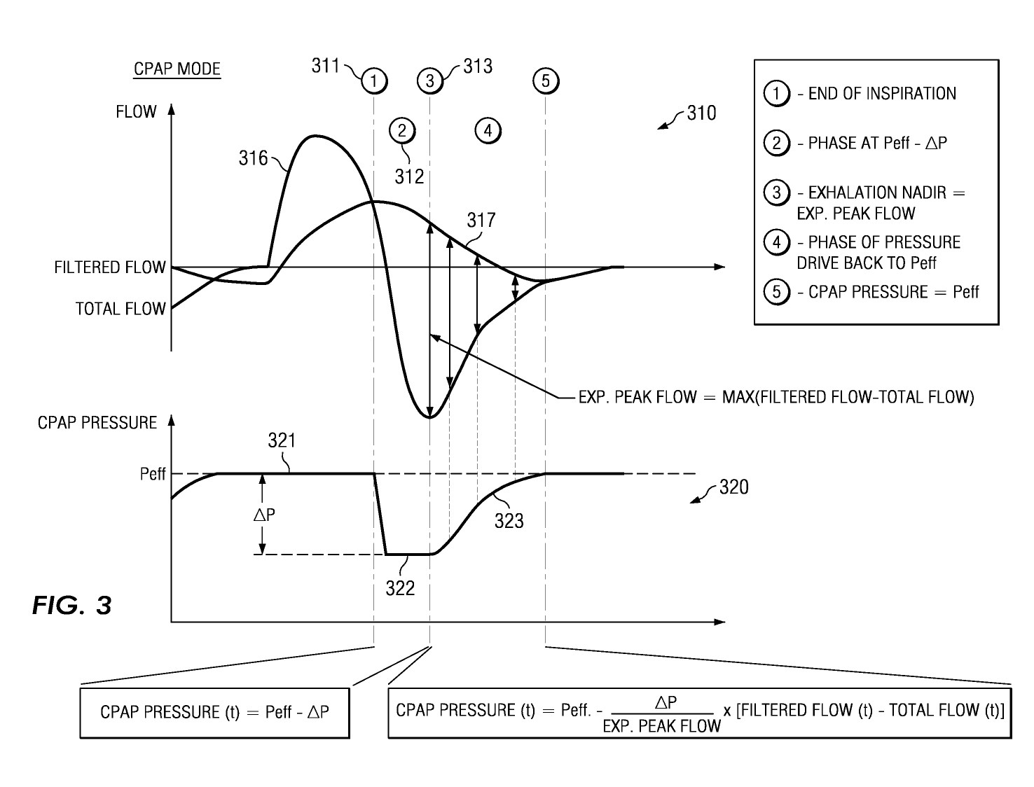

FIG. 3 is a graphical illustration of the application of an embodiment of the present disclosure for a subject undergoing CPAP therapy. The upper graph 310 of FIG. 3 shows the respiratory flow of a subject over time. Both the total flow 316 and the filtered flow 317 are shown. In the upper graph 310, the horizontal-axis represents time and the vertical-axis represents a flow rate. The total flow and filtered rates are used to determine the phases of inhalation and exhalation experienced by the subject using systems and methods known in the art. The lower graph 320 shows the pressure being applied to a subject over time. The horizontal-axis of the lower graph 320 represents time and the vertical-axis represents the pressure being applied to a subject.

According to an embodiment of the present disclosure, an effective pressure level 321 is applied to a subject during inhalation. This pressure can be delivered through mask 110 and provided by gas source 150 controlled by pressure controller 160. This higher positive pressure level may improve the ability of the subject to inhale. This effective pressure level is shown at point 321 in the lower graph. As inhalation is about to end at point 311, a relief pressure 322, e.g., a fixed value relief pressure, is applied from the beginning of exhalation until the nadir of exhalation 313. The near immediate application of a fixed relief pressure 322 value during exhalation, as opposed to the gradual application of a reduced pressure, offers a more immediate improvement to a subject's ability to exhale and reduces the likelihood of the occurrence of an apneic event. In a short time period before inhalation ends, the pressure applied to the subject is reduced as instantaneously as possible to a relief pressure, e.g., a relief pressure value 322. The effective pressure 321 begins to drop in a short time period near the end of inhalation 311 so that the relief pressure may be applied immediately or as soon as possible when exhalation begins This short period before inhalation ends represents the amount of time for the breathing device to lower the pressure to a relief pressure, e.g., to pressure value 322. This period may be less than three hundred milliseconds when an effective pressure is ten cmH2O and a predetermined pressure level is 3 cmH20. A relief pressure, e.g., relief pressure value 322, may be a fixed pressure value and may be applied to the subject until the nadir or peak of exhalation 313. The near immediate application of a relief pressure value, according to this example a fixed pressure, during exhalation, as opposed to a gradual application of a relief pressure, offers more immediate improvement to a subject's ability to exhale. After the nadir of exhalation 313, the applied pressure may be raised at a rate, e.g., a predetermined rate 323, such that an effective pressure may be reached when inhalation begins. This treatment may be applied throughout a subject's breathing.

Numerous other changes, substitutions, variations, alterations, and modifications may be ascertained to one skilled in the art and it is intended that the present invention encompass all such changes, substitutions, variations, alterations, and modifications as falling within the scope of the appended claims.

What is claimed is:

1. A method for delivering pressurized gas to an airway of a subject comprising:
applying a constant pressure at a level less than an effective pressure at or approximately at the beginning of exhalation until the nadir or approximately the nadir of exhalation;
raising the applied pressure to an effective pressure at a rate beginning at or approximately at the nadir of exhalation until the beginning or until approximately the beginning of inhalation; and
applying effective pressure during inhalation.

2. A method according to claim 1, wherein the effective pressure is prescribed by a physician.

3. A method according to claim 1, wherein the pressure level less than an effective pressure is about 3 cmH2O.

4. A method according to claim 1, wherein the rate comprises one of a predetermined rate and a rate proportional to a flow rate from the subject.

5. A method according to claim 1, further comprising determining the nadir of exhalation by measuring a flow rate from the subject and applying a low pass filter to the flow rate.

6. A method according to claim 1, wherein the beginning of exhalation is determined by measuring a low frequency component of a measured flow rate.

7. A system for delivering pressurized gas to an airway of a subject comprising:
a gas source;
a flow sensor to monitor a respiratory flow rate;
a filter operable to generate at least one of a filtered flow and a dynamic flow from the respiratory flow rate;
a pressure controller connected to the breathing device operable to control pressure levels applied from the gas source; and
an event detector connected to the pressure controller, the event detector operable to:
detect the beginning of exhalation in the subject;
provide a signal to the pressure controller to apply a pressure at a level less than an effective pressure;
detect the nadir of exhalation in the subject;
provide a signal to the pressure controller to raise the applied pressure to an effective pressure;
detect the beginning of inhalation; and
provide a signal to the pressure controller to apply pressure at an effective pressure.

8. A system according to claim 7, wherein the effective pressure is a physician prescribed pressure.

9. A system according to claim 7, wherein the pressure level less than an effective pressure comprises about 3 cmH2O or less.

10. A system according to claim 7, wherein the pressure controller is operable to direct the raise in the applied pressure to an effective pressure at a rate, said rate comprising at least one of a predetermined rate and a rate proportional to a flow rate from the subject.

11. A system according to claim 7, wherein the filter is operable to generate a filtered flow signal from the flow rate from the subject by measuring a low frequency component of the flow rate.

12. A system according to claim 7, wherein the filter is operable to generate a dynamic flow signal from the subject's flow rate by removing a low frequency component of the flow rate.

13. A computer-readable storage medium containing a set of instructions executable on a processor, the set of instructions comprising:
a routine operable to continuously monitor a respiratory flow rate from a subject; and
a routine operable to control a breathing gas generator operable to apply a constant pressure at a predetermined level less than an effective pressure at the beginning or at approximately the beginning of exhalation until the nadir or until approximately the nadir of exhalation, raise the applied pressure to the effective pressure beginning at the nadir or at approximately the nadir of exhalation until the beginning or until approximately the beginning of inhalation, and apply the effective pressure during inhalation.

14. A medium according to claim 13, wherein the effective pressure is a physician prescribed pressure.

15. A medium according to claim 13, wherein the predetermined level is about 3 cmH2O.

16. A medium according to claim 13, wherein the rate is one of predetermined rate and a rate proportional to a flow rate from the subject.

17. A medium according to claim 13, wherein the routine is further operable to filter the respiratory flow rate to generate a low frequency component of the flow rate.

18. A medium according to claim 13, wherein the routine is further operable to generate a dynamic flow from the respiratory flow rate by removing a low frequency component of the flow rate.

19. A system for delivering pressurized gas to an airway of a subject, said system comprising:
a gas generating means operable to deliver pressurized gas to the subject;
a flow sensing means operable to monitor a respiratory flow;
a filter means operable to generate at least one of a filtered flow and a dynamic flow from the respiratory flow rate;
a pressure controller means connected to the gas generating means operable to control pressure levels supplied by the gas generating means; and
an event detection means connected to the pressure controller means, wherein the event detection means is operable to:
detect the beginning or approximately the beginning of exhalation in the subject;
provide a signal to the pressure controller means to direct the gas generating means to supply a constant pressure at a level less than an effective pressure at the beginning of or at approximately the beginning of exhalation of the subject;
detect the approximate nadir or nadir of exhalation in the subject;
provide a signal to the pressure controller means to direct the gas generating means to raise the supplied pressure to an effective pressure at a predetermined rate upon detection of the approximate nadir or nadir of exhalation in the subject;
detect the beginning or approximate beginning of inhalation; and
provide a signal to the pressure controller means to direct the gas generating means to supply an effective pressure at the beginning or approximate beginning of exhalation.

20. A system for delivering pressurized gas to an airway of a subject, said system comprising:
a gas generating means operable to deliver pressurized gas to the subject;
a flow sensing means operable to monitor a respiratory flow;
a filter means operable to generate at least one of a filtered flow and a dynamic flow from the respiratory flow rate;
a pressure controller means connected to the gas generating means operable to control pressure levels supplied by the gas generating means; and
an event detection means connected to the pressure controller means, wherein the event detection means is operable to:
detect the beginning or approximately the beginning of exhalation in the subject;
provide a signal to the pressure controller means to direct the gas generating means to supply a constant pressure at a level less than an effective pressure at the beginning of or at approximately the beginning of exhalation of the subject;
detect the approximate nadir or nadir of exhalation in the subject;
provide a signal to the pressure controller means to direct the gas generating means to raise the supplied pressure to an effective pressure at a rate upon detection of the approximate nadir or nadir of exhalation in the subject, wherein the rate is proportional to the respiratory flow rate from the subject;
detect the beginning or approximate beginning of inhalation; and
provide a signal to the pressure controller means to direct the gas generating means to supply an effective pressure at the beginning or approximate beginning of exhalation.
